(19) United States
(12) Patent Application Publication
(10) Pub. No.: US 2012/0041276 A1
(43) Pub. Date: Feb. 16, 2012

(54) All in one medical monitor

(76) Inventors: Delcina Doreus, Inkster, MI (US); Evon Doreus, Inkster, MI (US)

(21) Appl. No.: 12/806,497

(22) Filed: Aug. 13, 2010

(51) Int. Cl. A61B 5/00

(52) U.S. Cl. 600/301

(57) ABSTRACT

The present invention provides a portable medical monitoring device designed for personal use to measure a variety of physical parameters, such as heart rate, blood pressure, oximetry (SpO2), and blood sugar (glucose) level. The device is attached to a belt or arm band that is secured around an individual when the device is being used and is equipped with a display screen, test select switches, an optical module and a processing module that analyzes data generated by the optical module.

### FIELD OF THE INVENTION

The present invention pertains to the field of medical monitors and more specifically to the field of personal medical monitors worn on the body.

### BACKGROUND OF THE INVENTION

The prior art has put forth several designs for medical monitoring devices worn on the body. Among these are:

U.S. Pat. No. 4,809,700 to Arrigo Castelli describes an armlet device with an electrode for measuring physiological electrical potentials.

U.S. Pat. No. 4,889,132 to 9 J. Stanford Hutcheson et al describes a portable automated blood pressure and heart rate measurement system.

U.S. Pat. No. D395,513 to Dietrich Rogler is a design patent covering the appearance of a portable medical monitor with display.

U.S. Pat. No. D420,446 to Bruce A. Kehr, et al is a design patent covering the appearance of a hand held portable medical monitor for managing the administration of medical and medical treatment regimens.

None of these prior art references describe the present invention.

### SUMMARY OF THE INVENTION

It is an object of the present invention to provide an improved portable medical monitoring device for monitoring vital signs such as heart rate, blood pressure, blood sugar level and oxygen content of red blood cells, for example.

### BRIEF DESCRIPTION OF THE DRAWING

FIG. 1 is a perspective view of the present invention.

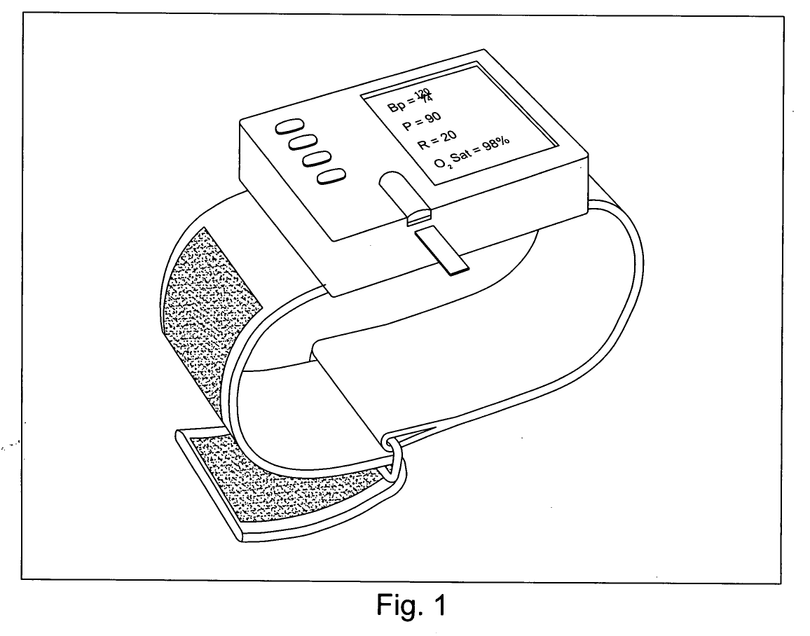

### DETAILED DESCRIPTION OF THE INVENTION

It is important to monitor vital signs because cyclical changes occur in the human body continuously. Events or conditions that alter normal cycles can interfere with many body functions and induce or facilitate disease. Vital signs including blood pressure, heart rate, basal body temperature, body-fat content and weight are good indicators of the cyclical and non-cyclical changes that are occurring in the body.

Daily monitoring of the vital signs can help assess the body's general health condition. Graph the vital signs, and the trends become apparent; analysis becomes faster and more accurate. When a health condition exists, such as hypertension, asthma, diabetes, or heart failure, graphing the vital signs with other factors such as blood glucose or peak-flow meter readings, while comparing the data to answers to related questions such as asthma, produces an excellent tool for managing health conditions.

Many individuals utilize a blood pressure monitor and the millions of individuals who suffer from COPD (chronic obstructive pulmonary disease and chronic bronchitis and emphysema) and those suffering from asthma and pulmonary fibrosis, frequently use a digital oximeter to measure SpO2. SpO2 is a measurement of the amount of oxygen attached to the hemoglobin cells in the circulatory system.

Another type of medical monitor, the glucose meter is used by people with diabetes to check their blood sugar at home, school, work, and play. All of these devices including blood pressure monitors, digital oximeters and glucose meters are used by millions of people in the U.S. and elsewhere, many using two or even three of the devices. Using multiple monitoring devices is inconvenient and time consuming.

The present invention, hereinafter referred to as the All in One, is an improved medical monitoring device, which is capable of measuring heart rate, blood pressure, oximetry (SpO2), and blood sugar (glucose) level.

The All In One is a portable medical monitoring device designed for personal use. The All In One is attached to a belt or arm band that is secured around an individual when the device is being used. The All In One is equipped with a display screen, test select switches, an optical module and a processing module that analyzes data generated by the optical module. The optical module typically includes first and second light sources (e.g., light-emitting diodes, or LEDs) that transmit optical radiation and infrared light.

The optical module also features a photo detector that detects radiation transmitted or reflected by an underlying artery. Typically, infrared LEDs sequentially emit radiation that is partially absorbed by blood flowing in the artery. The photo detector is synchronized with the LEDs, to detect transmitted or reflected radiation. In response, the photo detector generates a separate radiation-induced signal for each wavelength. The signal varies in a time-dependent manner as each heartbeat varies the volume of arterial blood and hence the amount of transmitted or reflected radiation. A microprocessor in the pulse oximeter processes the relative absorption of red and infrared radiation to determine the oxygen saturation in the patient's blood. In addition, the microprocessor analyzes time-dependent features to determine the patient's heart rate and blood pressure.

The All In On is also equipped with a glucose meter that is used for determining the approximate concentration of glucose in the blood. The All In One is an innovative and much needed medical monitoring device that can beneficially used by millions of individuals who need to monitor their physiological functioning.

Although this invention has been described with respect to specific embodiments, it is not intended to be limited thereto and various modifications which will become apparent to the person of ordinary skill in the art are intended to fall within the spirit and scope of the invention as described herein taken in conjunction with the accompanying drawings and the appended claims.

1. A personal medical monitoring device comprising means for measuring heart rate, blood pressure, oxygen content of red blood cells, and blood sugar level.

2. The medical monitoring device of claim 1, wherein the device comprises means for attachment to a user's belt.

3. The medical monitoring device of claim 1, wherein the device comprises an arm band for attachment to a user's arm.

4. The medical device of claim 1 comprising a display screen, test select switches, an optical module and a processing module for analysis of data generated by the optical module wherein the optical module typically includes first and second light sources that transmit optical radiation and infrared light, and further wherein the optical module comprises a photo detector that detects radiation transmitted or reflected by an underlying artery.

5. The medical device of claim 4 wherein the optical module comprises light emitting diodes (LEDs) capable of emitting light in the infrared portion of the electromagnetic spectrum capable of sequentially emitting radiation that is partially absorbed by blood flowing in the artery.

6. The medical device of claim 4 further comprising a photo detector synchronized with the LEDs, to detect transmitted or reflected radiation and wherein, the photo detector generates a separate radiation-induced signal in response to the reflected radiation for each wavelength.

7. The medical device of claim 6 capable of generating a signal which varies in a time-dependent manner as each heartbeat varies the volume of arterial blood and hence the amount of transmitted or reflected radiation.

8. The medical device of claim 6 further comprising a microprocessor in the pulse oximeter for processing the relative absorption of red and infrared radiation to determine the oxygen saturation in the patient's blood.

9. The device of claim 6, further comprising a microprocessor for analysis of time-dependent features to determine the patient's heart rate and blood pressure.

10. The device of claim 6 further comprising a glucose meter for determining the approximate concentration of glucose in the blood.
